(19) United States
(12) Patent Application Publication
(10) Pub. No.: US 2007/0212426 A1
(43) Pub. Date: Sep. 13, 2007

(54) Composition and method of retarding viral activity and reducing viral replication

(76) Inventors: Matthias Rath, Cape Town (ZA); Aleksandra Niedzwiecki, San Jose, CA (US); Vadim Ivanov, Castro Valley, CA (US); Waheed Roomi, Sunnyvale, CA (US); Raxit Jariwalla, San Jose, CA (US)

Correspondence Address: ALI KAMAREI ESQ, 280 COLORADO AVE, PALO ALTO, CA, 94301, US

(21) Appl. No.: 11/373,547

(22) Filed: Mar. 10, 2006

(51) Int. Cl. A61K 31/7048; A61K 31/401; A61K 31/353; A61K 31/198; A61K 33/34; A61K 33/32; A61K 33/06

(52) U.S. Cl. 424/638; 514/27; 514/456; 514/474; 514/562; 514/565; 514/566; 424/639; 514/423; 424/682

(57) ABSTRACT

A composition comprising of polyphenols, an ascorbic compound, lysine, and proline, have been found to retard viral activity. As such, methods of treating viral infection, and methods of retarding viral activity are provided herein.

### FIELD OF THE INVENTION

This invention relates to compositions and methods for treating health conditions caused by a viral infection and methods for retarding viral activity and viral replication. More particularly, this invention relates to treatment and/or prevention of health conditions caused by the influenza and avian influenza viruses.

### BACKGROUND OF THE INVENTION

Influenza is one of the oldest and most common infections. It poses a serious health problem causing significant morbidity and mortality, and imposing substantial economic costs. To date there are no effective antiviral therapies. Two classes of drugs are approved for influenza treatment: M2 ion channel blockers, such as amantadine and its derivative rimantadine, and Neuraminidase (NA) inhibitors. Antiviral drugs such as oseltamivir and zanmivir, block the function of neuraminidase protein, retarding virus from budding from the host. However, these drugs have their own limitation. Amantadine and rimantadine are synthetic drugs and as such can be toxic. Oseltamivir and zanamavir are less toxic than amantadine and rimantadine; however, none of these drugs are absolutely efficacious. In other words, none of these drugs can completely prevent the replication and spread of the flu viruses. As such, together with the toxicity they pose to the host, they are not particularly desirable.

Accordingly, methods and compositions, in particular those using micronutrients, are needed for retarding viral activity and reducing viral replication as well as improved methods for treating health conditions caused by viral infections.

### SUMMARY OF THE INVENTION

The present invention is based, at least in part, on the discovery that a composition comprising lysine, proline, arginine, an ascorbic compound, and polyphenols will retard viral activity and reduce viral replication thereby treating or retarding health conditions caused by a viral infection.

Accordingly, in one aspect, the invention provides methods of retarding or treating health condition caused by a viral infection by administering a composition comprising lysine, proline, arginine, an ascorbic compound, and polyphenols in an effective amount.

In one embodiment, the composition is comprised of lysine, proline, arginine, ascorbic acid, calcium, magnesium, polyphenols, N-acetyl-cysteine, selenium, copper, and manganese. In another embodiment, the composition is comprised of approximately 400-1500 mg lysine, 500-1500 mg proline, 200-1000 mg arginine, 400-1500 mg of ascorbic compound, 5-50 mg calcium, 10-100 mg magnesium, 500-2000 mg polyphenols, 100-500 mg N-acetyl-cysteine, 5-60 μg selenium, 0.5-5 mg copper, and 0.5-3 mg manganese. In a preferred embodiment, the composition is comprised of approximately 1000 mg lysine, 750 mg proline, 500 mg arginine, 710 mg of ascorbic compound, 22 mg calcium, 50 mg magnesium, 800 mg polyphenols, 200 mg N-acetyl-cysteine, 30 μg selenium, 2 mg copper, and 1 mg manganese.

In another embodiment, the composition is provided in the form of a tablet, a pill, an injection, an infusion, an inhalation, or a suppository. In yet another embodiment, the composition is provided in a pharmaceutically accepted carrier and means of delivery.

In another aspect, the invention provides a method to retard viral activity and or to reduce viral replication comprising treating a virus with a composition comprising lysine, proline, arginine, an ascorbic compound, and polyphenols in an effective amount. In a preferred embodiment, the composition is comprised of lysine, proline, arginine, ascorbic compound, calcium, magnesium, polyphenols, N-acetyl-cysteine, selenium, copper, and manganese.

In yet another aspect, the invention provides a pharmaceutical composition comprising an anti-viral drug and a composition comprising lysine, proline, arginine, ascorbic acid, calcium, magnesium, polyphenols, N-acetyl-cysteine, selenium, copper, and manganese. In one embodiment, the pharmaceutical composition further comprises a pharmaceutically acceptable carrier.

### BRIEF DESCRIPTION OF THE DRAWING

FIG. 1 shows an effective decrease of Influenza A virus antigen production in Vero cells (monkey kidney cells)_after these cells were exposed to human influenza A virus with serum-free medium, containing Trypsin, supplemented with various concentrations of a composition.

FIG. 2 shows an effective decrease of Influenza A virus antigen production in Vero cells after these cells were exposed to human influenza A virus with serum-free medium, absence of Trypsin, supplemented with various concentrations of a composition.

FIG. 3 shows marginal decrease of Influenza A virus antigen production in Vero cells after these cells were exposed to human influenza A virus with serum-free medium supplemented with various concentrations of a ascorbic acid.

FIG. 4 shows the retardation effect of a composition on Influenza Virus A's Neuraminidase Activity.

FIG. 5 shows the non-retardation effect of an ascorbic acid compound at various concentrations on Influenza Virus A's Neuraminidase Activity.

FIG. 6 shows the results of the composition on MMP expression in uninfected Madin Darby canine kidney (MDCK) cells.

FIG. 7 shows the effect of the composition on MMP expression in the presence of PMA in uninfected MDCK cells.

FIG. 8 depicts the effect of the composition on MMP expression in influenza virus-infected MDCK cells.

FIG. 9 shows the effect of the composition on MMP expression in the presence of PMA in influenza-infected MDCK cells. PMA enhanced MMP-9 (monomer and dimer) expression in infected cells without affecting MMP-2.

FIG. 10 depicts the results of the composition on MMP expression in uninfected monkey kidney (Vero) cells.

FIG. 11 shows the effect of the composition on MMP expression in the presence of PMA in uninfected Vero cells.

FIG. 12 depicts the effect of the composition on MMP expression in influenza virus-infected Vero cells.

FIG. 13 shows the effect of the composition on MMP expression in the presence of PMA in influenza-infected MDCK cells.

### DETAILED DESCRIPTION OF THE INVENTION

The term “virus” used herein refers to any of a large group of submicroscopic agents that act as parasites and consist of a segment of DNA or RNA surrounded by a coat of protein. Because viruses are unable to replicate without a host cell, they are not considered living organisms in conventional taxonomic systems. They are described as “live” when they are capable of replicating and causing disease. Accordingly, the term “viral activity” refers to any state of being active or any energetic action or movement or liveliness of a virus. Accordingly, the term “viral replication” refers to any process by which genetic materials, a single-celled organism, or a virus reproduces or makes a copy of itself.

The term “Neuraminidase” as used herein refers to a hydrolytic enzyme that removes sialic acid from mucoproteins and is found chiefly in microorganisms of the respiratory and intestinal tracts. Neuraminidase breaks the bonds that hold new virus particles to the outside of an infected cell. Once the enzyme breaks these bonds, this sets free new viruses that can infect other cells and spread infection. The term “Neuraminidase inhibitors” as used herein refers to a inhibitor that blocks the Neuraminidase′ activity and prevent new virus particles from being released, thereby limiting the spread of infection.

The term “Vero cells” as used herein refers to the African green monkey kidney cell line which is a suitable system for the primary isolation and cultivation of influenza A viruses. It is also known that Vero cells are suitable for isolation and productive replication of influenza B viruses.

The term “infection” as used herein refers to the presence of a virus in or on a subject, which if replication of the virus was retarded or of the activity of the virus was reduced, would result in a benefit to the subject. Accordingly, the term “infection” refers to the presence of pathogens at any anatomical site of a human or animal.

The term “treating” as used herein refers to the administration of a compound to a subject for therapeutic purposes. The term “administration” includes delivery to a subject by any appropriate method which serves to deliver the drug to the site of the infection. The administration of drug can be oral, nasal, parental, topical, ophthalmic, or transdermal administration or delivery in the form of solid, semi-solid, lyophilized powder, or liquid dosage forms. The dosage forms include tablets, suppositories, pills soft elastic and hard gelatin capsules, powders, solutions, suspensions, or aerosols, or the like, preferably in unit dosage forms suitable for simple administration of precise dosages.

The term “antiviral drug” as used herein includes any medication used specifically for treating viral infections. Specific antiviral drugs are used for specific viruses. Destroying or retarding the growth and reproduction of viruses. The term “antiviral drug” also includes any drugs used to retard viral infections. The drugs act by interfering with a virus's ability to enter a host cell and replicate itself with the host cell's DNA. Some drugs reduce the virus's attachment or entry into the cell; others retard replication or prevent the virus from shedding the protein coat that surrounds the viral DNA.

The term “antiviral drug” therefore, includes ribavirin, available since the mid-1980s, is used to treat respiratory syncytial virus (RSV), a cause of severe childhood respiratory infections. The term “antiviral drug” also includes acyclovir, which that interferes with an enzyme critical to the growth of the DNA chain. Although not a cure, the drug lessens the frequency and severity of outbreaks. Acyclovir is also used to lessen the pain and speed the healing of herpes zoster. The term “herpes zoxter” refers to an infection of a ganglion (nerve center) with severe pain and a blisterlike eruption in the area of the nerve distribution, a condition called shingles. The term “antiviral drug” also includes oseltamivir and zanamivir, which are antiviral drugs, as neuraminidase inhibitor, used in the treatment and prophylaxis of both influenza A and influenza B. The term “antiviral drug” also includes amantadine and rimantadine, which are an orally administered medicine used to treat, and in rare cases prevent, type A influenza. When taken within one to two days of developing symptoms, rimantadine and amantadine shorten the duration and moderate the severity of influenza.

The term “an agent” includes a plurality of agents, including mixtures thereof.

As used herein, the term “ascorbic compound” includes any pharmaceutically acceptable salt of ascorbate, including sodium ascorbate, as well as calcium ascorbate, magnesium ascorbate, ascorbyl palmitate and ascorbic acid itself and a combination thereof. The term “lysine” refers to lysine in its electrically neutral form or a pharmaceutically acceptable salt of lysine which includes lysine hydrochloride, lysine dihydrochloride, lysine succinate, lysine glutamate, lysine orotate as well as I-lysine. The term “proline” refers to proline and proline in a pharmaceutical acceptable salt of proline which includes proline hydrochloride, proline glutamate, as well as I-proline. The term “arginine” refers to arginine and arginine in a pharmaceutical acceptable salt of arginine which includes arginine hydrochloride, arginine glutamate, as well as I-arginine. The term “polyphenols” includes, but is not limited to, Epigallocatechin Gallate (EGCG), epicatechin gallate, epigallocatechin, epicatechin, and catechin which is an anti-oxidant polyphenol isolated from green tea, Red Wine Polyphenols (RWP) derived from grapes, and polyphenols derived generally from fruits and vegetables.

As used herein, the term “pharmaceutically acceptable salt” refers to those salts which retain the biological effectiveness and properties of the active ingredient of the biochemical composition, which are not otherwise undesirable. Pharmaceutically acceptable salts include, but are not limited to, salts of sodium, potassium, calcium, magnesium, aluminum and the like.

As used herein, the term “an effective amount” refers to that an amount of the biochemical composition disclosed in this application that when administered to an individual subject in need thereof, is sufficient to reduce the virus activity and/or growth thereby enhancing the antiviral activity.

As used herein, the term “therapeutically effective amount” refers to an amount of biochemical composition disclosed in this application that when administered to a human subject in need thereof, is sufficient to effect treatment for disease states caused by infection. A therapeutically effective amount may be determined routinely by one of ordinary skill in the art in view of this disclosure and the knowledge in the art. For example, one could start doses at levels lower than that required to achieve a desired therapeutic effect and gradually increase the dosage until the desired effect is achieved. In general, a suitable daily dose of a composition of the invention will be the amount of biochemical composition that is the lowest dose effective to produce a therapeutic effect. Such an effective dose will generally depend on the factors described above. It is preferred that administration be intravenous, intracoronary, intramuscular, intraperitoneal, or subcutaneous. Generally, a therapeutically effective daily dose is not less than about 10% and not more than about 200% of the amounts of individual ingredients listed in Table 1. Most preferably, a therapeutically effective daily dose is about 100% identical with the list of components in Table 1.

The term, “pharmaceutically acceptable means” as used herein means a pharmaceutically-acceptable material, composition or vehicle, such as a liquid or solid filler, diluent, excipient, solvent or encapsulating material, involved in carrying or transporting the antimicrobial agents or compounds of the invention from one organ, or portion of the body, to another organ, or portion of the body without affecting its biological effect. Each carrier should be “acceptable” in the sense of being compatible with the other ingredients of the composition and not injurious to the subject.

Additional embodiments and advantages of the invention will be set forth in the description that follows, and in part will be obvious from the description, or may be learned by practice of the invention. This invention may be realized and obtained by means of the composition and method of treatment particularly pointed out from the description and drawings, and from the claims.

The preferred route of administration is oral, using a convenient daily dosage regimen which can be adjusted according to the degree of severity of the disease state to be treated. For such oral administration, a pharmaceutically acceptable composition containing the compounds of the invention, or a pharmaceutically acceptable salt thereof, is formed by the incorporation of any of the normally employed, pharmaceutically acceptable excipients, such as, pharmaceutical grades of mannitol, lactose, starch, pregelatinized starch, magnesium stearate, sodium saccharine, talcum, cellulose ether derivatives, glucose gelatin, sucrose, citrate, propyl Gallate, and the like. Such compositions take the form of solutions, suspensions, tablets, pills, capsules, powders, sustained release formulations and the like.

Preferably such compositions will take the form of capsule, caplet, or tablet and therefore will also contain a diluent such as lactose, sucrose dicalcium phosphate, and the like; a disintegrant such as croscarmellose sodium or derivatives thereof; a lubricant such as magnesium stearate and the like; and a binder such as a starch, gum acacia, polyvinylpyrrolidone, gelatin, cellulose ether derivatives, and the like.

A composition of biochemical substances can contain at least one ascorbic compound selected from the group consisting of ascorbic acid, pharmaceutically acceptable ascorbate salts, calcium ascorbate, magnesium ascorbate, ascorbyl palmitate and/or mixtures thereof in combination with lysine hydrochloride, I-lysine or pharmaceutically acceptable lysine salts, I-proline, proline hydrochloride or pharmaceutically acceptable proline salts, or pharmaceutically acceptable argine salts, polyphenols thereof and/or mixtures of these compounds. The composition can be effective in reducing the antimicrobial resistance of microbes.

Preferably, a composition of biochemical substances comprises about 400 mg to about 1500 mg of L-Lysine. More preferably, a composition of biochemical substances comprises about 700 mg to about 1200 mg of L-Lysine. Most preferably, a composition of biochemical substances comprises about 1000 mg of L-Lysine.

Preferably, a composition of biochemical substances comprises about 500 mg to about 1500 mg of L-Proline. More preferably, a composition of biochemical substances comprises about 600 mg to about 1000 mg of L-Proline. Most preferably, a composition of biochemical substances comprises about 750 mg of L-Proline.

Preferably, a composition of biochemical substances comprises about 200 mg to about 1000 mg of L-Arginine. More preferably, a composition of biochemical substances comprises about 300 mg to about 800 mg of L-Arginine. Most preferably, a composition of biochemical substances comprises about 500 mg of L-Arginine.

Preferably, a composition of biochemical substances comprises about 400 mg to about 1500 mg of Ascorbic Acid. More preferably, a composition of biochemical substances comprises about 600 mg to about 1000 mg of Ascorbic Acid. Most preferably, a composition of biochemical substances comprises about 710 mg of Ascorbic Acid.

Preferably, a composition of biochemical substances comprises about 5 mg to about 50 mg of Calcium. More preferably, a composition of biochemical substances comprises about 10 mg to about 40 mg of Calcium. Most preferably, a composition of biochemical substances comprises about 22 mg of Calcium.

Preferably, a composition of biochemical substances comprises about 10 mg to about 100 mg of Magnesium. More preferably, a composition of biochemical substances comprises about 30 mg to about 70 mg of Magnesium. Most preferably, a composition of biochemical substances comprises about 50 mg of Magnesium.

Preferably, a composition of biochemical substances comprises about 500 mg to about 2000 mg of Polyphenols. More preferably, a composition of biochemical substances comprises about 600 mg to about 1200 mg of Polyphenols. Most preferably, a composition of biochemical substances comprises about 800 mg of Polyphenols.

Preferably, a composition of biochemical substances comprises about 100 mg to about 500 mg of N-acetyl-cysteine. More preferably, a composition of biochemical substances comprises about 150 mg to about 300 mg of N-acetyl-cysteine. Most preferably, a composition of biochemical substances comprises about 200 mg of N-acetyl-cysteine.

Preferably, a composition of biochemical substances comprises about 5 μg to about 60 μg of Selenium. More preferably, a composition of biochemical substances comprises about 10 μg to about 40 μg of Selenium. Most preferably, a composition of biochemical substances comprises about 30 μg of Selenium.

Preferably, a composition of biochemical substances comprises about 0.5 mg to about 5 mg of Copper. More preferably, a composition of biochemical substances comprises about 1 mg to about 3 mg of Copper. Most preferably, a composition of biochemical substances comprises about 2 mg of Copper.

Preferably, a composition of biochemical substances comprises about 0.5 mg to about 3 mg of Manganese. More preferably, a composition of biochemical substances comprises about 0.75 mg to about 2 mg of Manganese. Most preferably, a composition of biochemical substances comprises about 1 mg of Manganese.

Preferably, a composition of biochemical substances comprises L-Lysine, L-Proline, L-Arginine, Ascorbic Acid and Polyphenols. The composition of biochemical substances may be administered with a drug.

More preferably, a composition of biochemical substances comprises L-Lysine, L-Proline, L-Arginine, Ascorbic Acid, Calcium, Magnesium, Polyphenols, N-acetyl-cysteine, Selenium, Copper and Manganese and is administered with a drug.

Most preferably, a composition of biochemical substances consists essentially of L-Lysine, L-Proline, L-Arginine, Ascorbic Acid, Calcium, Magnesium, Polyphenols, N-acetyl-cysteine, Selenium, Copper and Manganese and is administered with an antiviral drug.

The composition may include a conventional pharmaceutically acceptable means or excipient and a compound of the invention as the active agent, and in addition, may include other medicinal agents, pharmaceutical agents, carriers, adjuvant, etc. These compositions may also contain additional agents, such as preservatives, wetting agents, emulsifying agents and dispersing agents.

Pharmaceutical compositions of the present invention may be administered to epithelial surfaces of the body orally, parenterally, topically, rectally, nasally, intravaginally, intracisternally. They are of course given by forms suitable for each administration route.

In some methods, the compositions of the invention can be topically administered to any epithelial surface. An “epithelial surface” according to this invention is defined as an area of tissue that covers external surfaces of a body, or that lines hollow structures including, but not limited to, cutaneous and mucosal surfaces.

Throughout this disclosure, various aspects of this invention can be presented in a range format. For example, description of a range such as from 1 to 6 should be considered to have specifically disclosed subranges such as from 1 to 2 to from 1 to 5, from 2 to 3 to from 2 to 5, etc., as well as individual numbers within that range, for example, 1, 2, 3, 4, 5, and 6. This applies regardless of the breadth of the range.

The practice of the present invention may employ, unless otherwise indicated, conventional techniques in various fields. Specific illustrations of suitable techniques can be had by reference to the examples herein below. However, other equivalent conventional procedures can, of course, also be used. Such conventional techniques and descriptions can be found in standard laboratory manuals such as Genome Analysis: A Laboratory Manual Series (Vols. I-IV), Using Antibodies: A Laboratory Manual, Cells: A Laboratory Manual, PCR Primer: A Laboratory Manual, and Molecular Cloning: A Laboratory Manual (all from Cold Spring Harbor Laboratory Press), Stryer, L. (1995) Biochemistry (4th Ed.) Freeman, New York, Gait, “Oligonucleotide Synthesis: A Practical Approach” 1984, IRL Press, London, Nelson and Cox (2000), Lehninger, Principles of Biochemistry 3^{rd} Ed., W.H. Freeman Pub., New York, N.Y. and Berg et al. (2002) Biochemistry, 5^{th} Ed., W.H. Freeman Pub., New York, N.Y., Short Protocols in Molecular Biology, 3rd Ed., ed. by Ausubel, F. et al. Wiley, N.Y. (1995), Methods In Enzymology Academic Press, Inc., N.Y., Immunochemical Methods In Cell And Molecular Biology Mayer and Walker, eds., Academic Press, London (1987), Handbook Of Experimental Immunology, Volumes I-IV D. M. Weir and C. C. Blackwell, eds. (1986), all of which are herein incorporated in their entirety by reference for all purposes.

The following specific examples are provided as a guide to assist in the practice of the invention, and are not intended as a limitation on the scope of the invention.

### EXAMPLES

### Example 1

This example illustrates the preparation of a representative composition containing the biochemical compounds listed in the following table (Table 1).

TABLE 1

| Biochemical Substances | Units | Amount |
|---|---|---|
| L-Lysine | mg | 1000 |
| L-Proline | mg | 750 |
| L-Arginine | mg | 500 |
| Ascorbic Acid | mg | 710 |
| Calcium | mg | 22 |
| Magnesium | mg | 50 |
| Polyphenols | mg | 1000 |
| N-acetyl-cysteine | mg | 200 |
| Selenium | μg | 30 |
| Copper | mg | 2 |
| Manganese | mg | 1 |

mg = milligram
μg = microgram

### Example 2

To demonstrate the utility of the composition of the invention as the therapeutic and preventive agents for health conditions caused by a viral infection whereby the composition effectively retard viral activity and viral replication, we evaluate the composition of these biochemical compounds for its ability to retard Influenza Virus A's Neuraminidase Activity and for its ability to retard Influenza A virus' nuclear production in Vero cells after Vero cells were exposed to Influenza A virus.

A two-day old, sub-confluent monolayers of Vero cells growing in 24-well plate (˜50,000 cells/well) were washed with PBS (phosphate buffer solution) and exposed to human influenza A virus (VR-1520) in serum-free medium (50 microliter/well) at 33° C. for one hour

The virus inoculum was left in and cells were fed serum-free medium (containing trypsin) supplemented with various concentrations of the composition containing the compounds listed in Table 1 as indicated above and incubated further at 33° C.

After 24 hours, an aliquot of culture supernatant was collected, mixed with an equal volume of diluent for lysis of virus antigen and assayed for the level of influenza A nuclear protein (NP) along with positive control (standards) using an enzyme immunoassay kit (MK120 from Takara Bio Inc.).

The data plotted above show the amount of Influenza A virus antigen (units/ml) detected in supernatants of cultures treated for 24 hours with various doses of the composition containing the compounds listed in Table 1.

Collectively, the results indicate that the compound was effective in retarding viral activity and viral replication as evidenced by the reduction in Influenza antigen production in Vero Cell.

The Influenza Antigen Production in Vero Cells is measured in Units per millimeter. The concentration of compound containing composition listed in Table 1 is listed is mcg/ml.

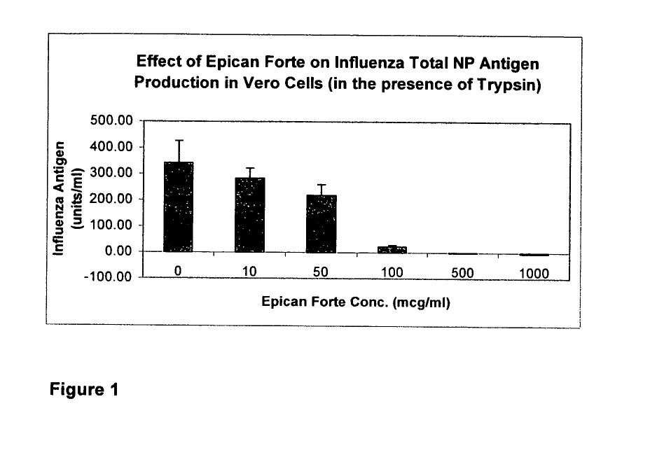

Results of the study, as illustrated in FIG. 1 demonstrate that the treatment of Vero cells inoculated with human influenza A virus with the composition affected the virus' activity and replication in the Vero cells and resulted in decreasing production of nuclear protein. As such, the production of Influenza antigen is greatly reduced. Accordingly, the results indicate that the composition is effective in retarding and reducing viral activity and viral replication.

FIG. 1 is a graph of result from a laboratory experiment conducted using Vero cell exposed to human influenza A virus for one hour and thereafter being treated with the biochemical composition containing the compounds listed in Table 1 for an additional 24 hours. This experiment was conducted in the presence of Trypsin, which is a proteolytic enzyme that generally promotes the growth of viruses. It is further accepted that Trypsin mimics or simulates the in vivo spread of the influenza virus in an in vitro environment. The change to the Influenza antigen production under various concentrations of the composition was recorded at the end of the 24 hours.

While the control, which did not include the composition listed in Table 1, exhibits an output of 245 antigen units/ml, inoculated Vero cells treated with 50, 100, 250, 500 and 1000 mcg/ml of the composition containing compounds listed in table 1 exhibited respectively at an output of antigen at 60%, 40%, 12%, 10% and 5% to the control's output. Accordingly, with each increasing dosage of the composition in Table 1, there was a significant reduction in the presence of the viral antigen, indicating less growth and less spread of the virus. A dose response curve is evident.

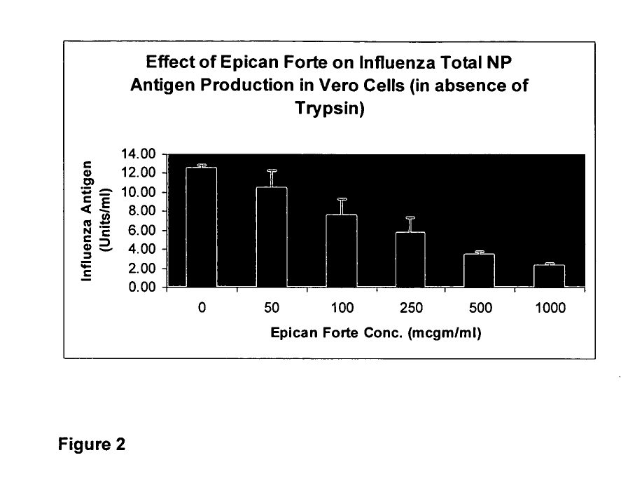

FIG. 2 is a different set of experiments run under the same conditions and methods as those in FIG. 1 except that it was done without the presence of Trypsin. As expected, viral activity without the growth promoting effects of Trypsin was significantly lower, approximately 12 antigen units/ml compared to 245 antigen units/ml in the presence of Trypsin, however, the composition of Table 1 in various concentrations exhibited a similar dose response curve, albeit a muted one.

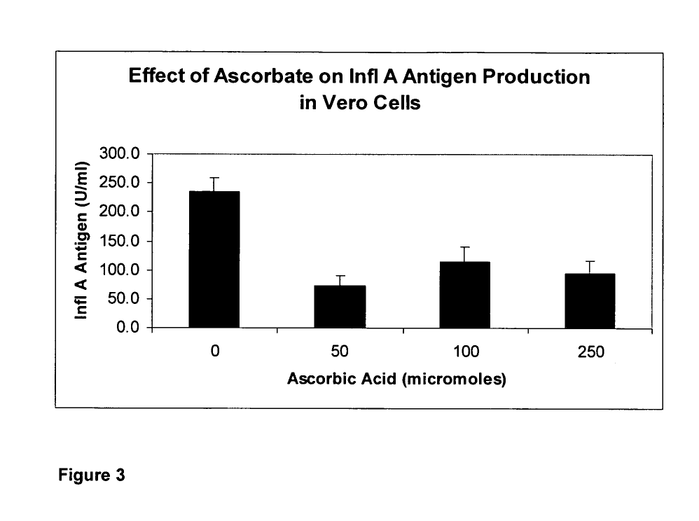

Vitamin C or Ascorbic Acid is generally though to aid in the prevention and/or treatment of influenza in humans. Accordingly, the effectiveness of the composition of Table 1 was compared to that of ascorbic acid. FIG. 3 is a graph of results from a laboratory experiment similar to the experiment conducted for the graph in FIG. 1 except that inoculated Vero cells were exposed to an ascorbic compound and not exposed to the biochemical composition containing the compounds listed in Table 1. While the control exhibits an output of 245 Antigen units/ml, inoculated Vero cells treated with 50, 100, and 250 micromoles of an ascorbic compound exhibited respectively at an output of Antigen of 35%, 45%, and 41% to the control's output.

FIG. 3 demonstrates that an ascorbic compound is not as effective in limiting viral activity of inoculated cell lines. It fact, the effectiveness to retard viral activity is not linear to the ascorbic compound concentration. The result indicates that the compound containing the compositions listed in Table 1 can be 7 times more effective in retarding the viral activity and viral replication than an ascorbic compound. As such, the composition listed in Table 1 is surprising in its effectiveness in retarding Influenza A's viral activity and in its ability to reduce Influenza A' viral replication.

### Example 3

Another set of in vitro experiments generally accepted to measure the effectiveness of antiviral drugs are experiments that measure Neuraminidase activity of a virus. Neuraminidase is an enzyme or protein that that is required by the virus, and generally exists within the virus particle, that is required by the virus for cell infectivity. That is Neuraminidase is required by the virus in order to spread. Specifically, Neuraminidase has functions that aid in the efficiency of virus release from cells. Neuraminidase cleaves terminal sialic acid residues from carbohydrate moieties on the surfaces of infected cells. This promotes the release of progeny viruses from infected cells. Neuraminidase also cleaves sialic acid residues from viral proteins, preventing aggregation of viruses. Administration of chemical inhibitors of neuraminidase is a treatment that limits the severity and spread of viral infections.

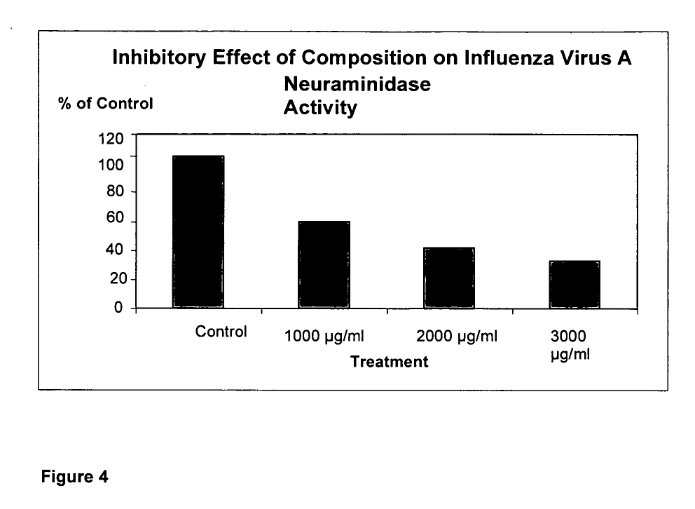

A higher Neuraminidase activity is associated with increase spread of the virus and lower Neuraminidase activity is associated with less spreading of the virus. Successful reduction of Neuraminidase protein can prevent the virus from budding from the host; thereby effectively retard the viral activity and viral replication. The method of these experiments are well known in the art, however, in brief, cell free virus are mixed with an equal volume of the composition in Table 1 or Ascorbic Acid in another, at various concentrations, and any inhibitory activity is observed at various time points after the mixture. Spectrophotometer assay for Neuraminidase has been established in the laboratory at using 4-methylumbelliferyl N-acetylneuramic acid as a substrate in the cell free system. Accordingly, FIG. 4 demonstrates laboratory experiments conducted to test the effectiveness of the composition of Table 1, as measured on its effectiveness on inhibiting Neuraminidase activity.

FIG. 4 is a graph of Neuraminidase activity of Influenza Virus A after the Influenza Virus A is treated with various concentration of the composition listed in Table 1. Here, the results show that the Influenza Virus A treated with 1000, 2000, and 3000 microgram/ml of the compositions listed in table 1 exhibited respectively at 58%, 38%, and 30% to the control's Neuraminidase activity. Accordingly, the compositions listed in table 1 are shown to be effective in retarding viral activity and viral replication by limiting Neuraminidase activity.

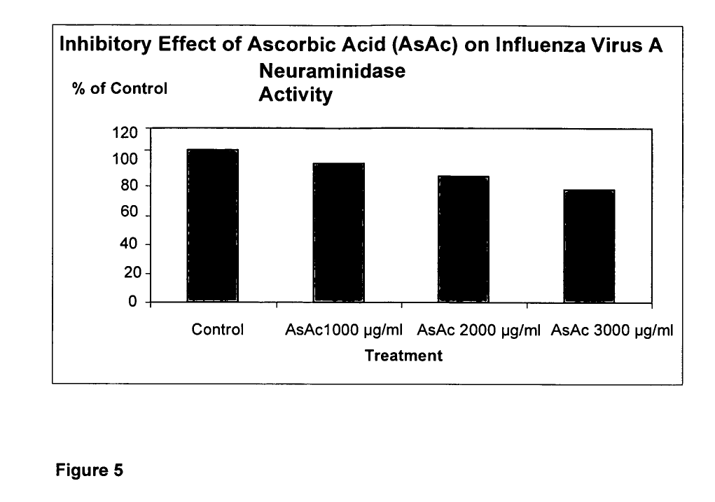

Likewise, the effectiveness of the composition of Table 1 to retard viral activity was compared to that of ascorbic acid. FIG. 5 is a graph of results from a laboratory experiment similar to the experiment conducted for the graph in FIG. 4 except Influenza Virus A is treated with various concentration of an ascorbic compound and not exposed to the biochemical composition containing the compounds listed in Table 1.

Here, the results show that the Influenza Virus A treated with 1000, 2000, and 3000 microgram/ml of an ascorbic acid exhibited respectively at 90%, 85%, and 80% to the control's Neuraminidase activity. Accordingly, the compositions listed in table 1 are shown to be effective in retarding viral activity and viral replication by limiting Neuraminidase activity.

FIG. 5 demonstrates that an ascorbic compound is not as effective in retarding viral activity and viral replication by limiting Neuraminidase activity. The result indicates that the compound containing the compositions listed in Table 1 can be almost 3 times more effective in limiting Neuraminidase activity. As such, the composition listed in Table 1 is surprising in its effectiveness in retarding Influenza A's Neuraminidase activity thereby retarding its viral activity and in its ability to reduce Influenza A' viral replication.

### Example 4

Matrix metalloproteinase's (MMPs) roles in various malignancies are well established; in particular are their implications in tumor invasion, metastasis, and spread of influenza virus. MMPs have the ability to digest basement membrane and ECM components. Basement membrane is mainly composed of type IV collagen and separates stomal and epithelial cells. For the virus to reach the stromal cells and blood vessels, the basemen membrane or ECM must be disrupted. MMP-2 and MMP-9 in particular have been shown to play an important role in the degradation of epithelial cell layer and ECM or basement membrane.

Accordingly, experiments were conducted to test the effectiveness of the composition of Table 1 and its effect on production of MMP-9. MDCK (Madin-Darby canine kidney) or Vero cells are infected with influenza virus as described above under experimental protocols for nuclear protein (NP) expression, and then exposed to various concentrations of the composition of Table 1 or ascorbic acid. To study enhanced expression of MMP-9, PMA (phorbol merystil acetate) is added to the incubation medium. At 24-48 hours post infection, the culture medium is collected and assayed for MMP expression by performing gelatinase zymography. The latter is carried out using 10% Novex pre-cast polyacrylamide gels in the presence of 0.1% gelatin. Culture media mixed with sample buffer are loaded on the gels and SDS-polyacrylamide gel electrophoresis (SDS-PAGE) is performed using Tris-glycine SDS buffer. After electrophoresis, the gels are washed with 2.5% Triton X-100 for 30 minutes (to remove SDS). The gels are then incubated overnight in substrate buffer and stained with 0.5% Coomasie Blue. Protein standards are run concurrently for determination of approximate molecular weights.

In the experiments described below, the test composition was tested at 0 (untreated control), 50, 100, 250, 500 and 1000 mcg/ml of the composition.

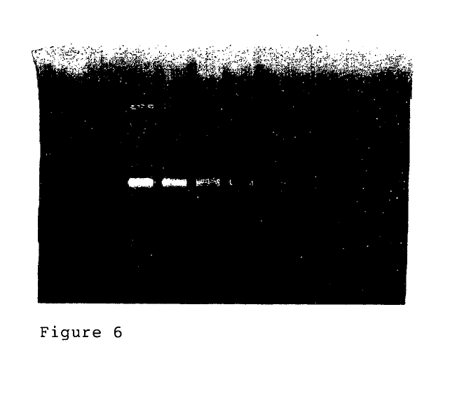

FIG. 6 depicts the results of the composition on MMP expression in uninfected Madin Darby canine kidney (MDCK) cells. Zymography showed only one band corresponding to MMP-9 (97 kilodaltons). The band intensity decreased with increasing concentration of the composition, showing a very faint band at the highest dose tested (1000 mcg/ml).

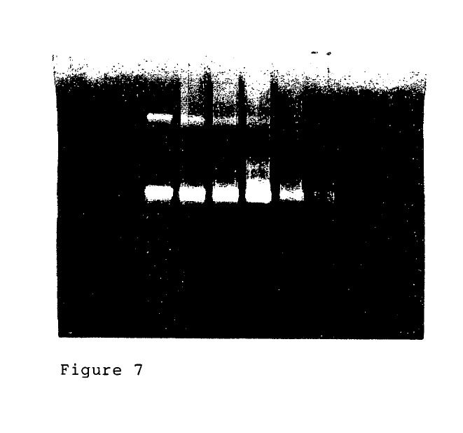

FIG. 7 shows the effect of the composition on MMP expression in the presence of PMA in uninfected MDCK cells. PMA enhanced the expression of MMP-9 and its dimer without affecting MMP-2 expression. Both monomer and dimer showed dose-dependent decrease with increasing concentration of the composition.

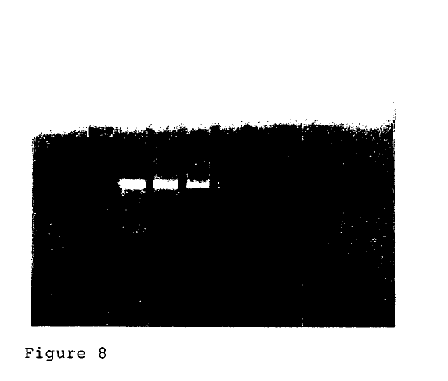

FIG. 8 depicts the effect of the composition on MMP expression in influenza virus-infected MDCK cells. Viral infection enhanced MMP-9 expression and in contrast to uninfected cells induced MMP-2 (68 kDa) protein expression. In the presence of the composition, the intensity of MMP-2 expression was inhibited partially at low concentration (50 mcg/ml) and totally at 100 mcg/ml, whereas MMP-9 showed dose-dependent decline with a faint band seen at 1000 mcg/ml.

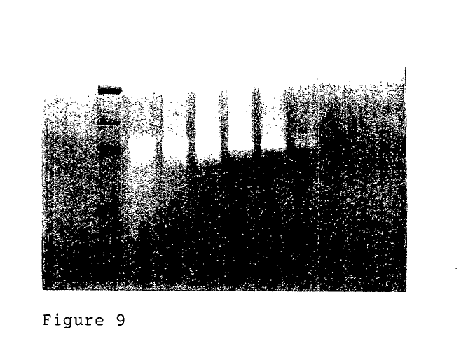

FIG. 9 shows the effect of the composition on MMP expression in the presence of PMA in influenza-infected MDCK cells. PMA enhanced MMP-9 (monomer and dimer) expression in infected cells without affecting MMP-2. In the presence of the composition, MMP-9 showed dose-dependent decrease, with a faint band seen at 1000 mcg/ml. MMP-2 was partially suppressed at 50 mcg/ml and totally suppressed at 100 mcg/ml of the test composition.

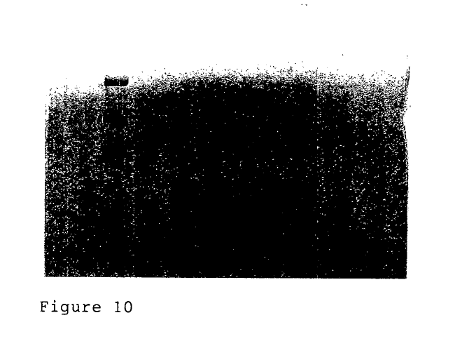

FIG. 10 depicts the results of the composition on MMP expression in uninfected monkey kidney (Vero) cells. In contrast to uninfected MDCK cells, Vero cells do not show expression of MMP-9 or MMP-2 proteins either in the absence or the presence of the composition.

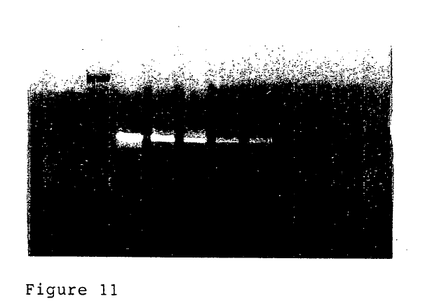

FIG. 11 shows the effect of the composition on MMP expression in the presence of PMA in uninfected Vero cells. PMA treatment enhanced the expression of MMP-9 and its dimer without affecting MMP-2 expression. In the presence of the composition, MMP-9 expression was reduced in a dose-dependent manner.

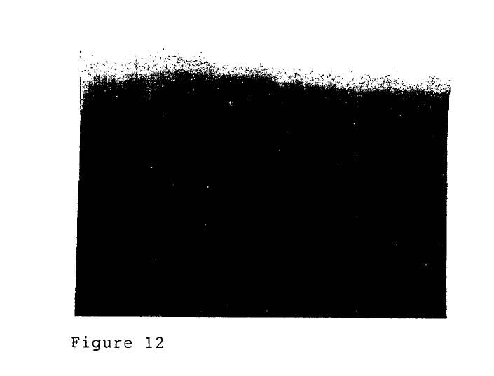

FIG. 12 depicts the effect of the composition on MMP expression in influenza virus-infected Vero cells. In contrast to uninfected Vero cells, infected cells showed two distinct bands corresponding to MMP-9 and MMp-2 expression. The composition exhibited suppression of both metallo-proteinases in a dose-dependent fashion.

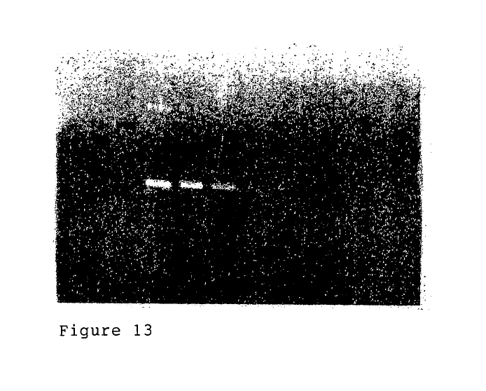

FIG. 13 shows the effect of the composition on MMP expression in the presence of PMA in influenza-infected MDCK cells. Upon PMA treatment, Vero cells showed a large increase in MMP-9 and its dimer. This expression was inhibited by the test composition in a dose-dependent manner. PMA did not affect MMP-2 activity. In the presence of the composition, MMP-2 expression was inhibited in a dose-dependent fashion with full suppression at 250 mcg/ml.

As such, the composition listed in Table 1 is surprising in its effectiveness in retarding viral activity, because of reduced viral replication, growth and ability to spread. The retardation effect of the composition listed in Table 1 on Influenza Virus A's Neuraminidase activity and on Influenza Antigen production in Vero Cells demonstrate a significant therapeutic effect of the biochemical composition and a retardation of the influenza virus and on its disease effect. Accordingly, based on the experimental results, one of skill in the art would recognize that application of a biochemical compound in accordance with the invention will be effective in the treating and retarding health conditions caused by a viral infection. The above Examples clearly show that composition of Table 1 is effective in the reduction of virus activity within host cells and can be used for the treatment of viral infections.

An example of the biochemical compound listed in Table 1 is Epican Forte™, as mentioned in US patent application publication no. 2004/0242504 which is fully incorporated herein by reference.

While the present invention has been described with reference to specific embodiments thereof, it should be understood by those skilled in the art that various changes may be made and equivalents may be substituted without departing from the true spirit and scope of the invention. One of ordinary skill in the art would appreciate that the effective amounts of the biochemical compounds may vary depending on the variations in patients, durations of treatment etc. Modifications may be made to adapt a particular situation, and composition of matter. A number of embodiments of the invention have been described in the present application; nevertheless, it will be understood all such modifications are intended to be within the scope of the following claims.

The contents of all references, pending patent applications and published patents, cited throughout this application are hereby expressly incorporated by reference. It is to be understood that the above description is intended to be illustrative and not restrictive. Those skilled in the art will recognize, or be able to ascertain using no more than routine experimentation, many equivalents to the specific embodiments of the invention described herein. Such equivalents are intended to be encompassed by the following claims.

What is claimed is:

1. A method of retarding or treating a health condition caused by a viral infection comprising administering a composition comprising lysine, proline, an ascorbic compound, and polyphenols, wherein the composition is administered in therapeutically effective amounts to retard viral activity and or to reduce viral replication.

2. A method of retarding or treating a health condition caused by a viral infection comprising administering a composition comprising of lysine, proline, arginine, an ascorbic compound, calcium, magnesium, polyphenols, N-acetyl-cysteine, selenium, copper, and manganese, wherein the composition is administered in therapeutically effective amounts to retard viral activity and or to reduce viral replication.

3. A method of retarding or treating a health condition caused by a viral infection comprising of administering a composition comprising about 400 to about 1500 mg lysine, about 500 to about 1500 mg proline, about 200 to about 1000 mg arginine, about 400 to about 1500 mg of ascorbic compound, about 5 to about 50 mg calcium, about 10 to about 100 mg magnesium, about 500 to about 2000 mg polyphenols, about 100 to about 500 mg N-acetyl-cysteine, about 5 to about 60 μg selenium, about 0.5 to about 5 mg copper, and about 0.5 to about 3 mg manganese.

4. A method of retarding or treating a health condition caused by a viral infection comprising administering a composition comprising about 1000 mg lysine, about 750 m proline, about 500 mg arginine, about 710 mg of ascorbic compound, about 22 mg calcium, about 50 mg magnesium, about 800 mg polyphenols, about 200 mg N-acetyl-cysteine, about 30 μg selenium, about 2 mg copper, and about 1 mg manganese.

5. The composition of biochemical substances according to claim 1-4. wherein the lysine is selected from the group consisting of lysine, I-lysine, lysine hydrochloride, and/or a pharmaceutically acceptable salt thereof.

6. The composition of biochemical substances according to claim 1-4. wherein the proline is selected from the group consisting of proline, I-proline, proline hydrochloride, and/or a pharmaceutically acceptable salt thereof.

7. The composition of biochemical substances according to claim 2 and 4, wherein the arginine is selected from the group consisting of arginine, I-arginine, arginine hydrochloride, and a pharmaceutically acceptable salt thereof.

8. A method as in one of claims 1-4 in which the composition is in the form of tablets, pills, injections, infusions, inhalations, suppositories or other pharmaceutically acceptable means of delivery.

9. A method of retarding viral activity and or reducing viral replication comprising treating a virus with a composition comprising lysine, proline, arginine, ascorbic compound, polyphenols, wherein the composition is administered in therapeutically effective amounts to retard viral activity and or reducing viral replication.

10. A method of retarding viral activity and or reducing viral replication comprising treating a virus with a composition comprising lysine, proline, ascorbic compound, calcium, magnesium, polyphenols, N-acetyl-cysteine, selenium, copper, and manganese, wherein the composition is administered in therapeutically effective amounts to retard viral activity and or reducing viral replication.

11. A pharmaceutical composition comprising of an antiviral drug and a further composition comprising lysine, proline, arginine, an ascorbic compound, calcium, magnesium, polyphenols, N-acetyl-cysteine, selenium, copper, and manganese, wherein the composition is administered in therapeutically effective amounts to retard viral activity and or to reduce viral replication.

12. The composition of biochemical substances according to claim 1-4, wherein the polyphenols are selected from the group consisting of Epigallocatechin gallate (EGCG) and other catechins selected from the group consisting of Epigallocatechin gallate, Gallocatechin gallate, Epicatechin gallate, Catechin gallate, Epigallocatechin, Catechin, Epicatechin and/or a pharmaceutically acceptable salts thereof.
